(19) United States
(12) Patent Application Publication
(10) Pub. No.: US 2006/0249144 A1
(43) Pub. Date: Nov. 9, 2006

(54) Ultrasonic Aerosol Generator

(75) Inventors: Wesley H. DeHaan, Belmont, MA (US); Wiwik S. Watanabe, Mountain View, CA (US)

Correspondence Address: PATREA L. PABST;PABST PATENT GROUP LLP, 400 COLONY SQUARE, SUITE 1200, ATLANTA, GA, 30361, US

(73) Assignee: Pulmatrix Inc.

(21) Appl. No.: 11/381,952

(22) Filed: May 5, 2006

Related U.S. Application Data
(60) Provisional application No. 60/678,085, filed on May 5, 2005.
(60) Provisional application No. 60/715,670, filed on Sep. 9, 2005.

(51) Int. Cl. A61M 11/00

(52) U.S. Cl. 128/200.14; 128/200.16; 128/200.21

(57) ABSTRACT

An ultrasonic aerosol generator for delivering a liquid formulation in an aerosolized form at a high output rate of greater than 0.5 mL/minute, preferably greater than 1.0 mL/minute, and with diameters in a respirable size range, methods of using this device and kits including the device are described herein. The ultrasonic aerosol generator (10) contains at least (a) a liquid reservoir/aerosolization chamber (11), (b) a piezoelectric engine (12), (c) a relief aperture (13), and (d) an aerosol delivery element (20). Preferably the aerosolized particles that are delivered to the user through the aerosol delivery element have an average aerodynamic diameter of between 1 and 20 μm, more preferably between 1 and 10 μm, and most preferably between 1 and 5 μm. Optionally, the ultrasonic aerosol generator is designed to deliver more than one formulation simultaneously, preferably a low cost and/or stable formulation is administered simultaneously with a more expensive and/or labile formulation. In the preferred embodiment, the ultrasonic aerosol generator is a hand-held device designed for a single user.

### CROSS-REFERENCE TO RELATED APPLICATIONS

This application claims priority to U.S. Ser. No. 60/678,085, filed May 5, 2005 and U.S. Ser. No. 60/715,670, filed Sep. 9, 2005.

### FIELD OF THE INVENTION

The present invention is in the field of improved devices for aerosolizing and administering liquid formulations to end users.

### BACKGROUND OF THE INVENTION

In some applications there is a need to deliver high quantity of aerosolized formulations in a respirable particle size range over a short period of time, for example, for delivering active agents to prevent the spread of airborne respiratory infectious diseases (also known as “ARIDs”), or for treating cystic fibrosis. The currently available aerosol generators include nebulizers and humidifiers.

Liquid nebulization is a common method of medical aerosol generation. There are two types of nebulizers, jet and ultrasonic. The nebulizers are typically small, hand-held devices. Jet nebulizers use the Venturi principle to draw liquid up to a high velocity air jet, where the liquid is sheared to form small droplets. The energy for the high velocity air jet is supplied by an air compressor, which drives the operation. Ultrasonic nebulizers convert alternating current to high-frequency acoustic energy, which turns the solution into a very fine mist that is then gently expelled. Ultrasonic nebulizers typically contain a small drug reservoir designed to contain about 5 mL or less of liquid. Standard ultrasonic inhalers have the drug reservoir separated from the piezoelectric disc by a liquid medium and a non-porous typically plastic layer. They also contain a fan to force the aerosol out of the aerosolization chamber. Examples of standard ultrasonic nebulizers include MabisMist™ II hand held ultrasonic nebulizer and DeVilbiss™ PULMOSONIC® Ultrasonic Nebulizer. Some ultrasonic nebulizers contain a vibrating screen which is in contact with the drug solution and results in the formation of fine aerosol droplets. Examples of vibrating screen ultrasonic nebulizers include Pari GmbH eFlow and Nektar Aeroneb Go. Other ultrasonic nebulizers contain a stationary screen, with a vibrating horn in contact with the drug solution. The vibrating horn forces the drug solution through the stationary screen, resulting in the formation of fine aerosol droplets. Examples of stationary screen ultrasonic nebulizers include OMRON® MICRO AIRE® and I-Neb Adaptive Aerosol Delivery System (RESPIRONICS, INC.®). The jet and ultrasonic nebulizers currently available typically have low aerosol output rates, such as less than 0.5 mL/min.

Humidifiers are used to maintain humidity levels in closed environments. Ultrasonic humidifiers generate a water aerosol without raising its temperature. An electronic oscillation is converted to a mechanical oscillation using a piezoelectric disk immersed in a reservoir of mineral-free water. The mechanical oscillation is directed at the surface of the water, where the ultrasonic frequency creates a very fine mist of water droplets. Different ultrasonic humidifiers are described in U.S. Pat. No. 4,238,425 to Matsuoka et al., U.S. Pat. No. 4,921,639 to Chiu, and U.S. Pat. No. 6,511,050 to Chu. Some of these devices are designed to allow the water level of the storage tank to remain level or to allow the water tank to be refilled more efficiently. U.S. Pat. No. 6,793,205 to Eom describes a combined humidifier that is capable of completely sterilizing bacteria in the mist prior to spraying the mist to the atmosphere. However, these humidifiers are not designed for direct inhalation of the mist, nor are they designed to produce aerosol with particles in the respirable size range.

Therefore it is an object of the invention to provide improved devices for delivering large amounts of aerosolized formulations for pulmonary administration of liquid formulations.

It is a further object of the invention to provide an improved method of aerosolizing liquid formulations.

### BRIEF SUMMARY OF THE INVENTION

An ultrasonic aerosol generator for delivering a liquid formulation in an aerosolized form at a high output rate of greater than 0.5 mL/minute, preferably greater than 1.0 mL/minute, and with diameters in a respirable size range and methods of using this device are described herein. The ultrasonic aerosol generator (10) contains at least (a) a liquid reservoir/aerosolization chamber (11), (b) a piezoelectric engine (12), (c) a relief aperture (13), and (d) an aerosol delivery element (20). Preferably the aerosolized particles that are delivered to the user through the aerosol delivery element have an average aerodynamic diameter of between 1 and 20 μm, more preferably between 1 and 10 μm, and most preferably between 1 and 5 μm. Optionally, the ultrasonic aerosol generator is designed to deliver more than one formulation simultaneously, preferably a low cost and/or stable formulation is administered simultaneously with a more expensive and/or labile formulation. In the preferred embodiment, the ultrasonic aerosol generator is a hand-held device designed for a single user.

### BRIEF SUMMARY OF THE DRAWINGS

FIG. 1A is a three-dimensional perspective view of a cross-sectional view of one embodiment of the device.

FIG. 1B provides the same cross-sectional view of this embodiment with arrows showing the flow path for the liquid and the aerosol.

FIG. 1C provides the same cross-sectional view of this embodiment showing some of the optional features of the device.

FIG. 2 is a schematic of a hand-held ultrasonic aerosol generator device.

FIG. 3 is a schematic of another embodiment of the device.

FIG. 4 is a schematic of an ultrasonic aerosol generator designed to deliver two or more formulations simultaneously.

FIG. 5 is a bar graph comparing the aerosol output rate delivered (mL/min) for different commercially available devices with the devices shown in FIGS. 2 and 3.

FIG. 6 is a bar graph comparing the mass mean diameter (μm) for the aerosol particles released from different commercially available devices with the devices shown in FIGS. 2 and 3.

### DETAILED DESCRIPTION OF THE INVENTION

I. Ultrasonic Aerosol Generator

The ultrasonic aerosol generator (10) described herein contains (a) liquid reservoir/aerosolization chamber (11), (b) a piezoelectric engine (12), and (c) a relief aperture (13), and (d) an aerosol delivery element (20).

The device is designed to produce a high output of aerosolized particles that have diameters within a respirable size range. As generally used herein “high output” means greater than 0.5 mL/min, preferably greater than 0.8 mL/min, more preferably greater than 1.0 mL/min, and most preferably greater than 2.0 mL/min.

Preferably the aerosolized particles have an average aerodynamic diameter of between 1 and 20 μm, more preferably between 1 and 10 μm, and most preferably between 1 and 5 μm. The aerodynamic diameter for smooth, spherical particles can be approximated using the following equation.
dₚₐ=dₚₛ√ρₚ (Eq. 1)

Where: dₚₐ=aerodynamic particle diameter (μm)
dₚ=physical or actual diameter (μm)
ρₚ=particle density (g/cm³).

The size of the particles can be measured by any suitable method. One suitable method includes a laser diffraction analysis instrument (e.g. Sympatec Helos/BF, Sympatec, Princeton, N.J.). The laser beam is directed into a measuring zone at which point particles diffract the parallel beams of light. A multi-signal detector measures the angle of diffraction and the light intensity and converts them into a particle size distribution. The optical concentration (Copt) is determined. The volume median diameter (d₅₀) and geometric standard deviation (GSD) values can then be calculated.

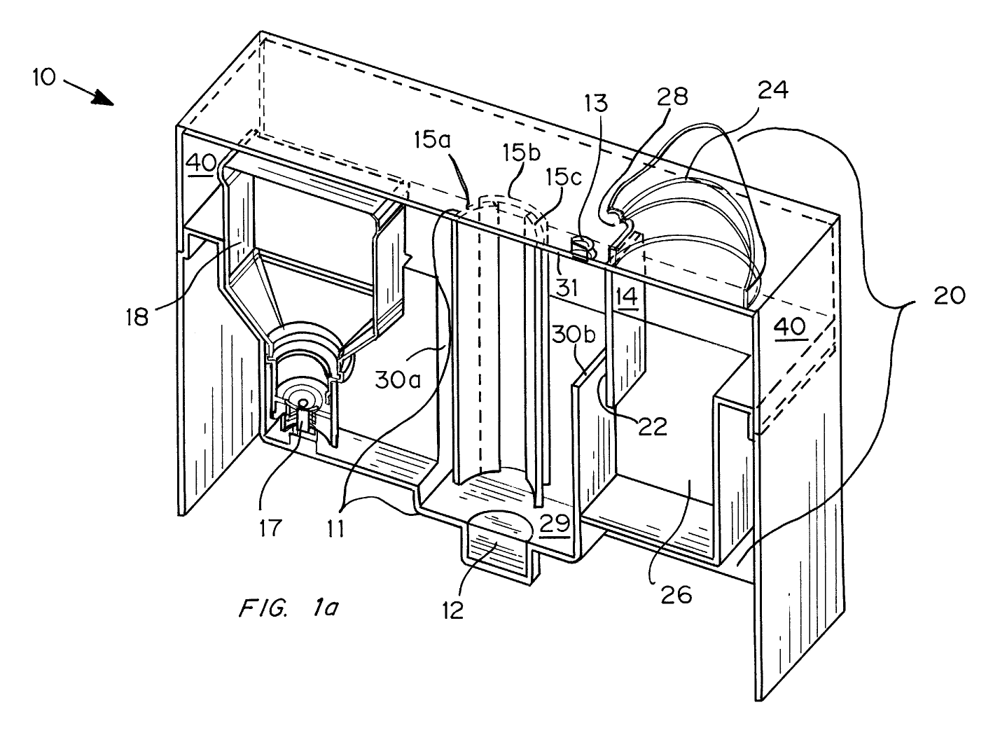

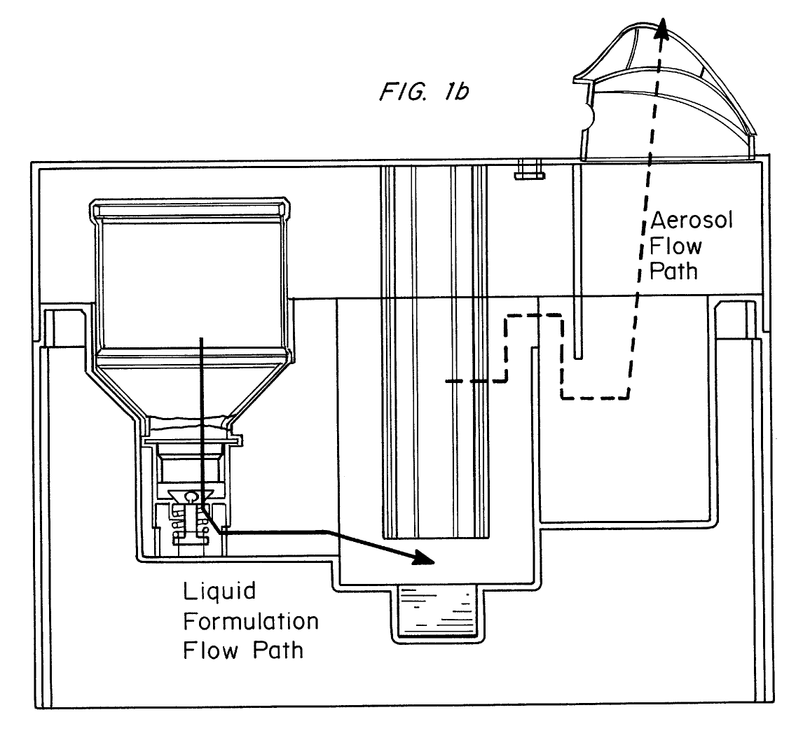

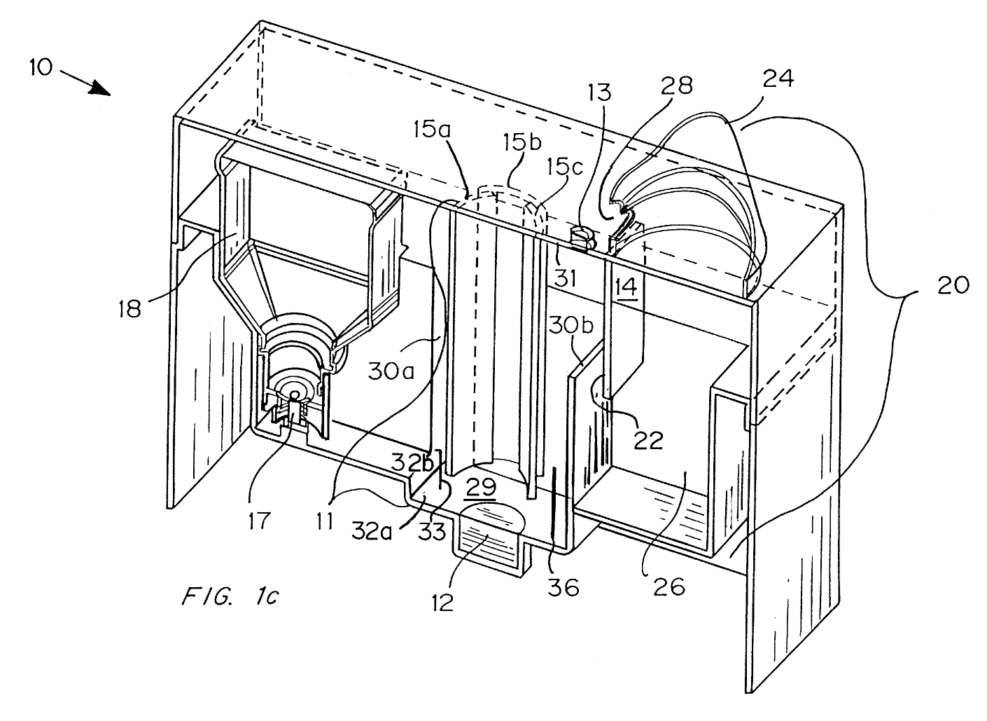

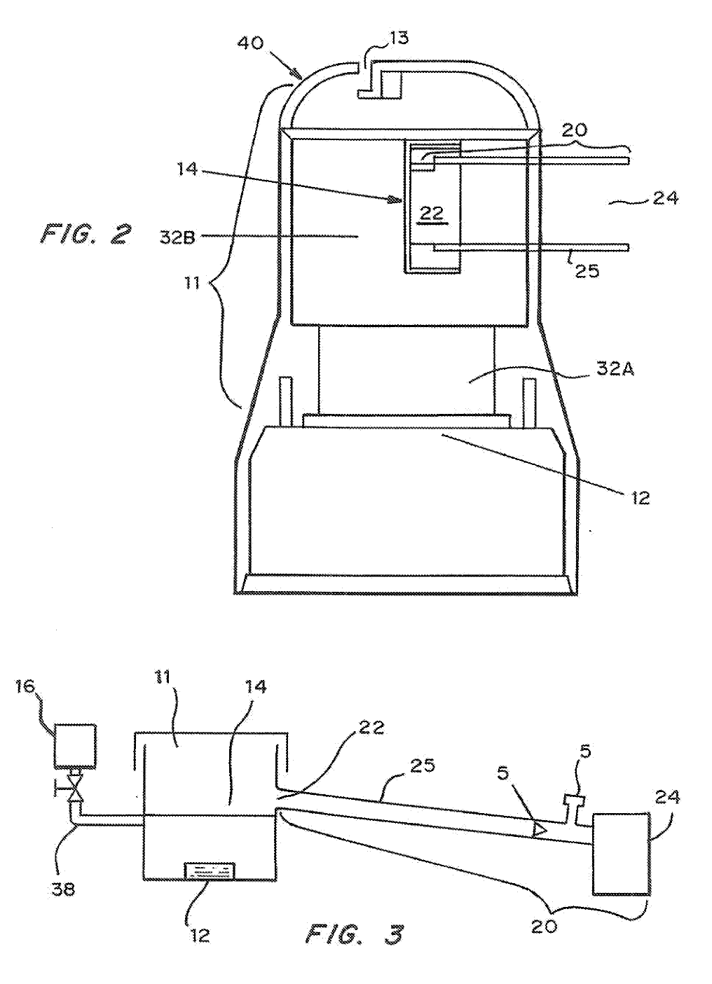

The ultrasonic generator may be a stationary device, such as in the form of a bench-top device, or may be portable, such as in the form a hand-held device. A preferred embodiment of the stationary device is shown in FIGS. 1A and 1B. A preferred embodiment of the hand-held device is shown in FIG. 2. The hand-held device is typically less than 5 inches in height and less than 4 inches wide. In the preferred embodiment, the hand-held device is approximately 4.5 inches tall and approximately 3 inches wide, at its widest point.

a. Liquid Reservoir/Aerosolization Chamber

The liquid reservoir/aerosolization chamber (also referred to herein as “the chamber”) (11) is a container with a bottom (29), one or more walls perpendicular to the bottom (30A and 30B) and a top (31). The reservoir is large enough to store at least 5 mL, preferably greater than 5 mL, more preferably greater than 8 mL, more preferably at least 15 mL, most preferably at least 45 mL of liquid formulation. In the stationary configuration, the reservoir is designed to contain preferably 50 to 300 mL of liquid, and most preferably 100 to 200 mL. In the hand-held configuration, the reservoir is designed to contain from 5 mL to 60 mL of liquid, preferably from 8 mL to 60 mL, and most preferably 15 to 45 mL. A typical dose delivers 1 mL of liquid formulation. Thus, the reservoir is typically designed to contain multiple doses. In contrast, conventional hand-held nebulizers have typically smaller reservoirs and only contain up to 5 mL of liquid. The piezoelectric engine (12) is typically located at the bottom of the reservoir so that it is in contact with the liquid formulation. The large volume in the liquid reservoir relative to conventional ultrasonic nebulizers allows for enhanced heat dissipation and sufficient formulation for multiple uses from single fill.

The liquid reservoir/aerosolization chamber contains two main regions, a lower region (32A) and an upper region (32B). The liquid is stored in the lower region (32A), and aerosol is formed in the upper region (32 B), circulated and released. The upper region (32 B) typically has a height, measured from the surface of the liquid formulation prior to turning on the device, of at least 20 mm, preferably 25 to 75 mm and most preferably 35 to 50 mm. The upper region is designed to contain a cone of aerosol generated when the piezoelectric engine is turned on. Typically a high wattage piezoelectric engine is used. The piezoelectric engine (12) is located in the lower region (32 A) of the chamber.

The chamber contains one or more outlets (22) (one is shown in FIGS. 1A and 1C) to which the one or more aerosol delivery elements can attach, directly or indirectly, such as through a connecting tube (25) (see FIGS. 2, 3, and 4). The location of the outlet(s) is optimized to allow gravity and the concentration gradient to transport the aerosol out of the chamber and into the aerosol delivery element. A blower or fan is not needed to transport the aerosol.

Optionally, the chamber (11) contains a thermometer (33) for measuring the temperature of the liquid formulation. Optionally, the device contains a switch (not shown in figure) that turns off the piezoelectric engine (12) if the temperature of the liquid formulation reaches a preset increased temperature. Optionally, the chamber (11) contains a temperature feed-back controller (not shown in figure) to maintain a preset temperature or temperature range during aerosolization.

Optionally the chamber (11) contains a liquid level sensor (36). Optionally, the device contains a switch (not shown in figure) that turns off the piezoelectric engine (12) if the level of the liquid reaches a preset minimum or maximum level.

1. Outlets

Aerosol outlet(s) are area(s) that connect the aerosolization chamber to the aerosol delivery element(s) and are typically located near the top of a wall that is perpendicular to the bottom of the chamber. As illustrated in FIGS. 1A and 1B, an outlet (22) is preferably located in the upper region of the chamber, distal to the piezoelectric engine (12). Preferably for the stationary devices, the outlet(s) (22) are greater than 20 mm above the surface of the piezoelectric engine (12) that is in contact with the liquid formulation, more preferably greater than 50 mm above the surface of the piezoelectric engine, and most preferably greater than 80 mm above the surface of the piezoelectric engine. The outlet (22) may be an opening, such as a hole, in one of the walls of the chamber. Alternatively, the outlet (22) may be in the form of a gap between a wall of the chamber and a baffle in the chamber, as shown in FIG. 1A. In this embodiment, the particles can only leave the chamber if they are able to pass over the wall and under the baffle (14), discussed below. Most large particles will not be able to make this turn, while the small particles with a diameter in the respirable size range will be able to pass through the outlet and into the aerosol delivery element.

As illustrated in FIG. 2, the chamber for the hand-held device typically contains one outlet (22). The outlet (22) may be an opening between the baffle (14) and the aerosol delivery element (20). In this embodiment, the particles can only leave the chamber if they are able to pass around the baffle (14) and through the narrow opening between the baffle (14) and the tube (25) for the aerosol delivery element (20). Most large particles will not be able to make this turn and fit through this space, while small particles with a diameter in the respirable size range will be able to pass through the outlet (22) and into the aerosol delivery element (20). Preferably for the hand-held device, the outlet (22) is located about 45 mm above the piezoelectric engine (12).

2. Baffles

Optionally, the reservoir contains one or more baffles (14) configured to direct the flow of the aerosol, filter out large particles, and therefore minimize aerosol deposition downstream of the chamber. The baffle may be of any suitable geometry including flat surface, cylindrical, perforated plate. Preferably, as shown in FIG. 1A, the baffle (14) is in the form of a wall. In another preferred embodiment for the hand-held device, as illustrated in FIG. 2, the baffle (14) is in the form of a cylinder. Preferably the diameter of the baffle is slightly larger than the diameter of the tube (25) for the aerosol delivery element. Typically particles that are greater than 30 microns, preferably greater than 20 μm in diameter, and more preferably particles that are greater than 10 μm in diameter, contact the baffle (14) and are removed from the aerosol flow. The baffle (14) is preferably designed to cause greater than 80% of particles that are greater than 30 micrometers in diameter, more preferably greater than 10 microns, to be removed from the airflow. Optionally, the removed particles are returned to the liquid in the reservoir (11).

A baffle is typically placed somewhere along the aerosol path. Preferably, as shown in FIG. 3, the baffle is located prior to any one-way exhalation valve (5) and most preferably it is located at the aerosolization chamber outlet (22). Optionally, the device contains more than one one-way valve, such as a series of two or more valves. The one or more one-way valves prevent the user from removing aerosolized particles from the device during exhalation.

Preferably, as shown in FIG. 1A, the chamber (11) contains a group of baffles (15A, 15B and 15C) that surround the particles as they are aerosolized. These baffles (15A, 15B and 15C) function as splash guards that catch particularly large particles and direct them to return to the liquid in the lower region.

3. Feeder

The liquid formulation can either be added directly to the liquid reservoir for aerosolization, or be added via a formulation feeder which allows the gradual addition of liquid. The feeder can be configured to control the liquid level in the liquid reservoir. The formulation feeder may be graduated, allowing the user to measure the amount of the formulation that is added. The feeder (element 16 in FIG. 3) may be the form of a tube (38) or the combination of a container and a tube. In a preferred embodiment, shown in FIG. 1A, the feeder contains a removable portion (17) that is designed to connect to a bottle (18) containing the liquid formulation and to connect with a tube (not shown). The opposite end of the tube connects with the liquid reservoir/aerosolization chamber (11), preferably with an opening in the lower region of the chamber (11). When connected to the feeder (16) the removable portion (17) allows the formulation to flow freely from the bottle to the liquid reservoir (11). The feeder (16) also contains means (not shown in Figures) for preventing the liquid from exiting the bottle (18) when the bottle is removed from the device. Suitable means include a valve or a plug. Optionally the formulation feeder is removable from the device to allow for the direct placement of the liquid formulation in the lower region of the chamber (32A).

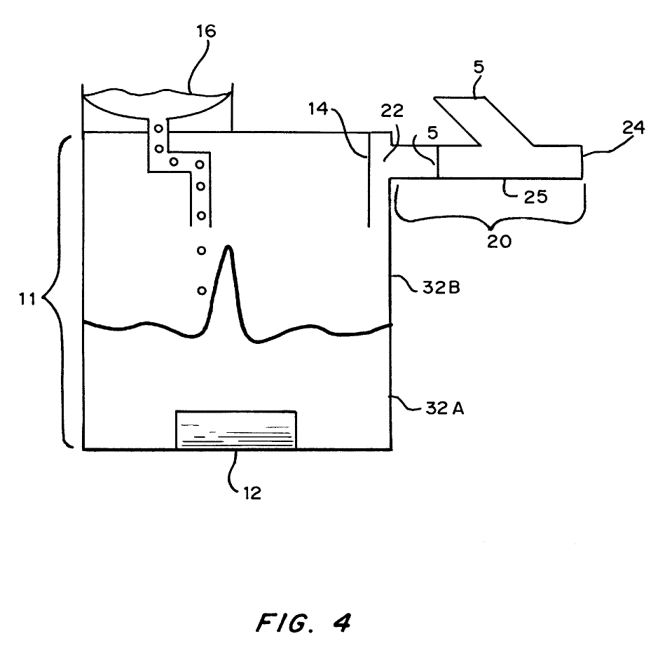

Optionally, the device may be designed to deliver more than one formulation simultaneously. This embodiment is particularly suitable for administering an expensive or labile formulation along with an inexpensive and/or stable formulation. In this embodiment, as shown in FIG. 4, one of the formulations may be added directly to the liquid reservoir while the second formulation is added via a formulation feeder (16). Alternatively, the liquid reservoir may contain two or more chambers, one for each formulation to be delivered. Alternatively, each of the formulations may be added separately to the liquid reservoir via separate formulation feeders. The formulation feeder may be graduated to allow the user to measure the amount of each formulation that is added to the liquid reservoir.

4. Membrane

In one embodiment, the reservoir contains a membrane that is designed to separate two liquids. In this embodiment, the piezoelectric engine (12) is in direct contact with a first liquid that is in contact through the membrane with a second liquid, i.e. the liquid formulation to be aerosolized. The membrane is preferably sufficiently non-porous to prevent contact between two liquids. The membrane is thin and may be formed of a synthetic or natural material (e.g. plastic or rubber).

This embodiment may be used to reduce or prevent heat transfer to the liquid formulation to be aerosolized. Preferably the liquid formulations that are delivered in this embodiment are heated by the piezoelectric engine when they are in direct contact with the engine and are unstable when heated.

Preferably the first liquid is selected to have the same impedence value as the liquid to be aerosolized, i.e. the second liquid. The first liquid is preferably water when the second liquid is an aqueous formulation.

b. Piezoelectric Engine

The piezoelectric engine (12) is typically a high wattage engine. Preferably the engine power is greater than 10 Watts, more preferably greater than 15 Watts, most preferably 25 to 35 Watts.

The ultrasound is preferably produced at a frequency greater than 100 kHz, more preferably greater than 1 MHz, most preferably greater than 1.5 MHz. Typical frequencies include 1.7 MHz and 2.4 MHz. An example of a suitable piezoelectric engine is one with a diameter of 20 mm, a frequency of 1.7 MHz, and a power of 24 Watts. Typically the piezoelectric engine has a flat surface in contact with the liquid.

c. Relief Aperture

In order to accomplish gravity driven flow of the aerosol, a relief aperture (13) open to the ambient air pressure is present in the chamber (11) (see e.g. FIGS. 1A, 2 and 3). This aperture (13) allows a small amount of airflow into the device to offset the vacuum created by the exiting aerosol, allowing a continuous aerosol flow out of the aerosol chamber (11). The aperture (13) is located at a height above that of the aerosol outlet(s) (22). Preferably the aperture (13) contains one or more baffles to prevent large aerosolized particles from escaping through it. Optionally, the top of the chamber (11) contains a removable lid (40), such as shown in the hand-held device illustrated in FIG. 2. When fully assembled, the lid attaches to the aerosolization chamber (32B). The relief aperture (13) may be located in the lid (see FIG. 1A). Optionally, the relief aperture (13) contains a one-way valve allowing inhalation, but preventing exhaled air from exiting through it (not shown in FIG. 2). Gravity driven flow of the aerosol, typically in combination with aerosol concentration gradients, forces the aerosol out of the chamber (11) and through the aerosol delivery element (20). Thus, the device does not contain a fan to force the aerosol out of the chamber (11).

d. Aerosol Delivery Element

The aerosol delivery element (20) contains an aerosol flow path from the outlet (22) to the end user(s). As shown in FIG. 2, the delivery element (20) may contain an aerosol exit tube (25), and user interface (24) to deliver the aerosol to the user. As shown in FIG. 1A, the delivery element may contain a standing reservoir for collecting the aerosol (26), and an exhalation vent (28) to minimize aerosol exposure to the ambient environment, in addition to a user interface (24). As shown in FIG. 3, the delivery element may also contain an exhalation one-way valve (5) to prevent formulation or device contamination during exhalation.

1. User Interface

The user interface (24) is designed to deliver the aerosol to the user. The user interface can be a mouthpiece, a mask that covers the user's mouth and nose and seals to the user's face, one nasal prong, two nasal prongs, or an opening that directs the aerosol to the user's mouth and/or nose when a user places his face within 15 cm, preferably within 5 cm of the opening. In the preferred embodiment, such as illustrated in FIG. 2, the device contains a single user interface, in the form of a mouthpiece. In another embodiment, the user interface is in the form of an opening (not shown in Figures). Optionally, the device contains more than one user interface to deliver the aerosolized formulation to more than one user, either concurrently or sequentially (not shown in Figures).

The location of the user interface does not need to be fixed relative to the outlet of the aerosolization chamber. In one embodiment, such as illustrated in FIG. 3, the user interface (24) is connected to the outlet with a flexible tube (25). In this embodiment, the user interface is preferably placed higher than the aerosol exit tube to prevent aerosol overflow in the aerosol delivery element. In another embodiment, such as where the user interface does not contain a flexible tube for connecting to the outlet, the user interface is preferably at least as high as the aerosol outlet from the chamber (see e.g. FIG. 2), more preferably above the highest point in the chamber (see e.g. FIG. 1A).

2. Aerosol Exit Tube

In one embodiment, such as shown in FIGS. 2 and 3, the user interface contains an aerosol exit tube (25) for connecting the user interface (24) to the outlet (22). The tube length is preferably less than 50 inches, more preferably less than 10 inches and most preferably less than 5 inches.

3. Standing Reservoir

In a preferred embodiment illustrated in FIG. 1A, the aerosol delivery element contains a standing reservoir (26) where the aerosol can accumulate prior to inhalation by the user. Preferably the standing reservoir volume is less than or equal to 500 mL, and most preferably less than or equal to 250 mL.

The bottom of the standing reservoir is typically located at a height that is equal to or below the height of the outlet from aerosolization chamber, preferably the bottom of the standing reservoir is located more than 20 mm below the outlet, and more preferably more than 50 mm below the outlet.

4. Exhalation Vent

In a preferred embodiment, the aerosol delivery element contains an exhalation vent (28) that opens to the surrounding environment during exhalation. Optionally, the vent includes a low resistance filter to minimize aerosol exposure to ambient air. This is particularly useful when the device is used in a clean room.

Optionally, the exhalation vent includes a one-way valve to minimize aerosol dilution by ambient air during inhalation. Optionally, the exhalation vent includes a second one-way valve, which closes during exhalation to direct the exhaled air through the exhalation vent and prevent both formulation contamination and the aerosol from being forced out of the device to the ambient during exhalation. Suitable valves may be formed of a thin, non-porous, lightweight material that is capable of maintaining its shape, such as a tightly woven nylon sheet, a single or multiple layer polymer film, or elastomer(s). The valves open and close with small pressure changes. In the preferred embodiment shown in FIG. 1A, preferably, the change in pressure is less than 10 cm of water, more preferably less than 1 cm water and most preferably less than 5 mm water. When it is in the open position, the valve should provide a large open area for flow of the aerosol through the valve to prevent aerosol deposition loss.

II. Method of Using the Device

The formulation to be administered is placed in the liquid reservoir, either by direct placement or by feeding the formulation to a formulation feeder which delivers the formulation to the liquid reservoir. Preferably a bottle (18) containing the formulation is connected to the formulation feed (17). This method of delivering the formulation reduces the risk of contamination of the liquid formulation.

Once the liquid reservoir is sufficiently filled with the formulation, the piezoelectric engine may be turned on. Preferably, the one or more aerosol delivery elements are attached to the one or more outlets prior to turning on the piezoelectric engine.

In one embodiment, the user places the user interface over his mouth and/or nose and begins breathing through the interface. In a second embodiment, the user places his mouth over the opening of the aerosol delivery element and begins breathing. One or more users may use the device simultaneously or sequentially.

In one embodiment, the device is used to administer more than one formulation simultaneously. In this embodiment, illustrated in FIG. 4, a first formulation, such as saline, may be placed directly in the lower region (32A) of the liquid reservoir/aerosolization chamber (11). The second formulation may be stored in a formulation feeder (16), preferably one with graduations to measure the amount of formulation added to the liquid reservoir (not shown in Figure). The outlet for the formulation feeder may be located above the surface of the first formulation (as shown in FIG. 4), such as in the upper region (32 B) of the chamber (11) or below the surface of the first formulation (not shown in FIG. 4). Alternatively, the device contains multiple formulation feeders (not shown in Figures) for adding the second formulation to the liquid reservoir from multiple locations, such as around the perimeter of the cone formed by the first formulation when the piezoelectric engine is turned on. Alternatively, the device may contain two compartments (not shown in Figures) in the lower region (32A) of the liquid reservoir/aerosolization chamber (11). The first formulation is added directly to the first compartment and the second formulation is added directly to the second compartment; preferably, the second compartment is configured to be above the first formulation. In the two compartment embodiment, the ultrasonic energy is delivered to the second compartment through the first formulation, this reduces the amount of heat transmitted to the second formulation.

In each of these devices, the ultrasonic energy is transmitted to both formulations and aerosolizes both formulations. Thus the aerosol is well-mixed prior to reaching the outlet (22) for the chamber. Further, the first formulation, typically a less expensive, more stabile formulation, may be used to rinse the walls of the device and conserve the second formulation, which is typically a more expensive formulation. This could allow for a higher emitted dose of the second formulation compared to devices administering the second formulation alone.

a. Liquid Formulations

The device may be used to deliver a liquid formulation to one or more users in settings such as a hospital, industrial, clean room, or home or personal setting. The liquid formulation may be in the form of a solution or suspension. Any liquid formulation that contains one or more excipients, optionally with one or more active agents may be administered using this device. Preferably the excipients contain one or more non-volatile salts. Preferably the formulation is an aqueous solution or suspension containing non-volatile components. In one embodiment, the formulation is physiological saline. The saline may be administered to act as an anti-infective agent. In other embodiments, the formulation contains an active agent, such as a drug. Suitable drugs include anti-viral, anti-bacterial and anti-microbial agent(s). The formulation preferably contains an aqueous solvent, but may contain one or more organic solvents. The solution is preferably stable at room temperature (25° C.), 37° C., 40° C., and/or greater than 60° C.

Optionally, the device may be designed to deliver more than one formulation simultaneously. For example, the device could deliver two formulations, where the first formulation is relatively inexpensive and stable, such as saline, and the second formulation is a more expensive and/or labile formulation. As generally used herein “more expensive” means that the second formulation is more expensive than the first formulation; typically the second formulation will cost at least 5 times the cost of the first formulation. Examples include saline as the first formulation and a drug formulation as the second formulation. Thus the second formulation may not be stable at room temperature and/or elevated temperatures, such as 37° C., 40° C., or greater than 60° C.

III. Uses for the Device

Preferably the device is used to deliver formulations that can suppress exhaled bioaersol production to prevent the spreading of ARID, or formulations for treatment and prevention of ARID (e.g. influenza, tuberculosis, or severe acute respiratory syndrome (SARS)). Typically, when the device is used to administer a single formulation at a time, the formulation will be a stable, aqueous formulation, such a saline, optionally containing one or more active agents, preferably the active agents are stable at greater than 40° C. and more preferably greater than 60° C. Optionally the device is used to administer a mixture of formulations. Optionally, the device may be used to deliver a second formulation which is less stable and/or more expensive than the first formulation.

Optionally, the device may be connected to another device, such as a ventilator or continuous positive airway pressure (CPAP).

### EXAMPLE

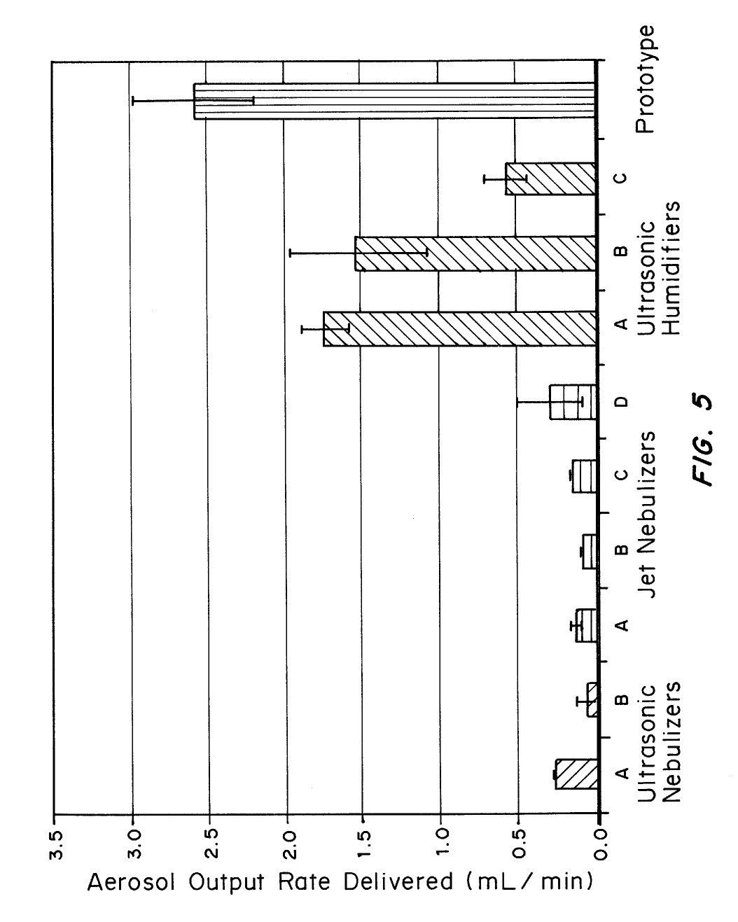

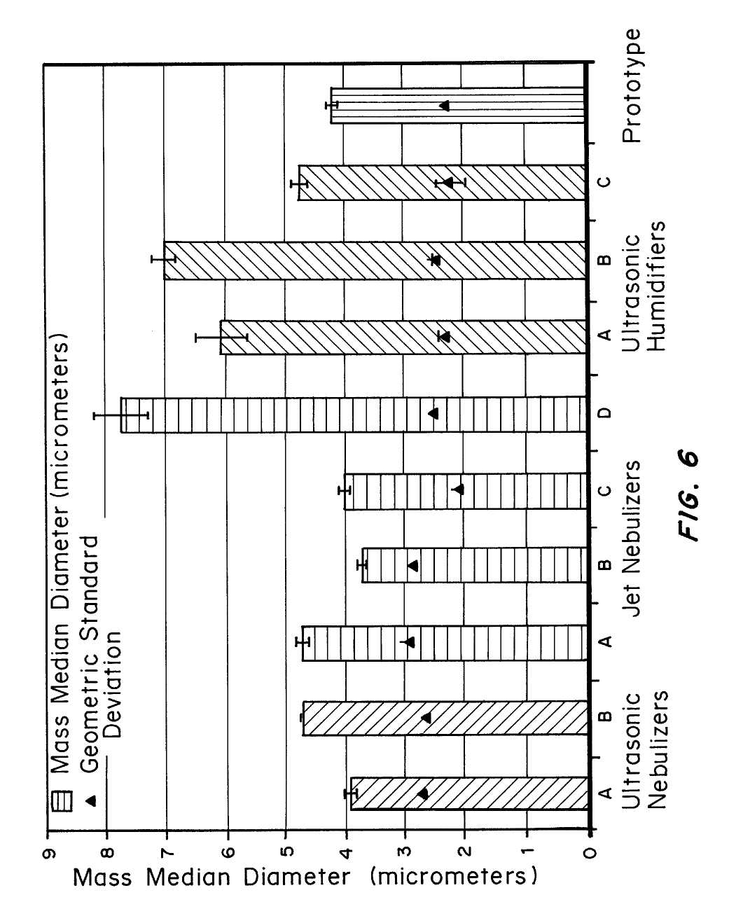

Two devices corresponding to the configurations depicted in FIGS. 3 and 2 respectively (labeled “Devices A and B”, respectively, in FIGS. 5 and 6) were tested and compared with commercially available ultrasonic nebulizers, jet nebulizers, and ultrasonic humidifiers. Two different ultrasonic nebulizers were tested (AERONEB® Go Model 7000 (AeroGen, Inc.) and OMRON® MICROAIRE® Model NE-U22V, labeled A and B, respectively, in FIGS. 5 and 6) four different jet nebulizers were tested (Hudson RCI Micro Mist Model 1882; Invacare SIDESTREAM® Model MS2400 (Medic-Aid Limited Corp., United Kingdom); RESPIRONICS® VENTSTREAM® Model PL273, and OMRON® CompAir Elite Model NE-C21V; labeled A, B, C, and D, respectively, in FIGS. 5 and 6), and three different ultrasonic humidifiers were tested (WALGREENS® Model 700, VICKS® Model V5100N and SUNBEAM® Model 697-6, labeled A, B and C, respectively, in FIGS. 5 and 6) to determine their aerosol output rates and average aerosolized particle size. All commercially available devices were utilized according to their operating instructions. Both the prototypes utilized piezoelectric engines operating at 1.7 MHz and 26 Watts with a 20 mm diameter piezoelectric disk. All tests were performed at room temperature and pressure with isotonic saline.

The amount of aerosol emitted by each device during one dosing period (i.e. the aerosol output rate) was determined gravimetrically, by placing two filters (303, Vital Signs) in series at the exit of the device and weighing the filters before and after actuation. Aerosol output rates were calculated from measurements of the change in weight of the filters. The tests were performed with 15 L/min of air drawn through the system for all nebulizers and the prototypes and sufficient airflow for the ultrasonic humidifiers to capture the output aerosol driven by the humidifiers' internal fan. The data is presented in FIG. 5. As shown in FIG. 5, the devices described in the specification and illustrated in FIGS. 2 and 3 (Devices A and B) had the greatest output rate of all of the devices tested, with an aerosol output rate of greater than 2.0 mL/min. All of the other devices had aerosol output rates of less than 2.0 mL/min. All of the jet nebulizers and ultrasonic nebulizers had aerosol output rates of less than 0.5 mL/min.

All particle sizing tests were performed using a Sympatec Helos laser diffraction analysis device with a R2 lens. The same test flow rates and device configurations were used for the particle size testing as for the aerosol output tests. Each device was activated and placed in front of the laser beam. The laser beam was directed into a measuring zone at which point particles diffract the parallel beams of light. A multi-signal detector measured the angle of diffraction and the light intensity and converted this data into a particle size distribution. The optical concentration (Copt) was determined. The mass median diameter (d₅₀) and geometric standard deviation (GSD) values were then calculated. The data is presented in FIG. 6. As shown in FIG. 6, the devices described in the specification and illustrated in FIGS. 2 and 3 (Devices A and B) produced particles within the respirable size range, with mass median diameters of about 4 μm.

Those skilled in the art will recognize, or be able to ascertain using no more than routine experimentation, many equivalents to the specific embodiments of the invention described herein. Such equivalents are intended to be encompassed by the following claims.

We claim:

1. An ultrasonic aerosol generator comprising
(i) a liquid reservoir/aerosolization chamber, wherein the chamber comprises an upper region and a lower region, wherein the upper region comprises one or more outlets,
(ii) a piezoelectric engine, wherein the piezoelectric engine is located in the lower region of the chamber,
(iii) at least one baffle, wherein the baffle is located in the upper region of the chamber;
(iv) a relief aperture, wherein the aperture is located above the outlet(s), and
(v) an aerosol delivery element,
wherein the generator can produce aerosolized particles in a respirable size range with an aerosol output rate of greater than 1 mL/minute.

2. The generator of claim 1, wherein the lower region of the chamber holds greater than 5 mL of liquid.

3. The generator of claim 1, wherein the piezoelectric engine is a high wattage engine.

4. The generator of claim 3, wherein the engine power is greater than 10 Watts.

5. The generator of claim 1, wherein the aerosol delivery element further comprises at least one element selected from the group consisting of an aerosol exit tube, an exhalation one-way valve, a standing reservoir, a user interface, and an exhalation vent.

6. The generator of claim 5, wherein the aerosol delivery element comprises a user interface selected from the group consisting of mouthpieces, masks, nasal prongs, and openings.

7. The generator of claim 6, wherein the aerosol delivery element further comprises a standing reservoir.

8. The generator of claim 7, wherein the standing reservoir has a volume less than or equal to 500 mL.

9. The generator of claim 1, wherein the generator is in the form of a hand-held device.

10. The generator of claim 9, wherein the generator comprises a user interface, wherein the user interface comprises a tube and a mouthpiece.

11. The generator of claim 10, wherein the upper region comprises one outlet and wherein the baffle is in the shape of a cylinder and is located at the outlet.

12. The generator of claim 1, wherein the lower region of the chamber is holds greater than 5 mL of liquid.

13. The generator of claim 1, further comprising at least one feeder, wherein the feeder is connected to the lower region of the chamber.

14. The generator of claim 1, further comprising at least one feeder, wherein the feeder is connected to the upper region of the chamber.

15. The generator of claim 14, comprising more than one feeder.

16. A method of using an aerosol generator for delivering a liquid formulation to a user, wherein the aerosol generator comprises
(i) a liquid reservoir/aerosolization chamber, wherein the chamber comprises an upper region and a lower region, wherein the upper region comprises one or more outlets,
(ii) a piezoelectric engine, wherein the piezoelectric engine is located in the lower region of the chamber,
(iii) at least one baffle, wherein the baffle is located in the upper region of the chamber;
(iv) a relief aperture, wherein the aperture is located above the outlet(s), and
(v) an aerosol delivery element,
wherein the generator can produce aerosolized particles in a respirable size range with an aerosol output rate of greater than 1 mL/minute,
wherein the method comprises
(a) filling the lower region of the chamber with the liquid formulation to be aerosolized and
(b) turning on the piezoelectric engine.

17. The method of claim 16, further comprising placing a user interface on top of or in close proximity to the user's mouth and nose prior to turning on the engine.

18. The method of claim 16, wherein the liquid formulation comprises one or more excipients or active agents, or a combination thereof.

19. The method of claim 16, wherein the liquid formulation is in the form of aqueous solution or suspension.

20. The method of claim 19, wherein the liquid formulation comprises one or more non-volatile salts.

21. The method of claim 20, wherein the liquid formulation comprises saline.

22. The method of claim 20, wherein the non-volatile salts comprise at least 0.1% by weight of the liquid formulation.

23. The method of claim 17, wherein the generator delivers a dose of at least 0.5 mL of the liquid formulation to the user.

24. The method of claim 16, further comprising adding a second liquid formulation to be aerosolized to the chamber prior to step (b).

25. The method of claim 24, wherein the second liquid formulation is placed in one or more feeders prior to adding it to the chamber.

26. The method of claim 24, wherein the second liquid formulation is added to the upper region of the chamber.

27. The method of claim 24, wherein the second liquid formulation comprises an active agent.

28. The method of claim 16, wherein the aerosol generator administers an effective amount of an aerosolized formulation to treat or prevent the spreading of airborne respiratory infectious diseases.

29. A kit comprising an aerosol generator and one or more liquid formulations, wherein the aerosol generator comprises
(i) a liquid reservoir/aerosolization chamber, wherein the chamber comprises an upper region and a lower region, wherein the upper region comprises one or more outlets,
(ii) a piezoelectric engine, wherein the piezoelectric engine is located in the lower region of the chamber,
(iii) at least one baffle, wherein the baffle is located in the upper region of the chamber;
(iv) a relief aperture, wherein the aperture is located above the outlet(s), and
(v) an aerosol delivery element,
wherein the generator can produce aerosolized particles in a respirable size range with an aerosol output rate of greater than 1 mL/minute.
